(19) United States
(12) Patent Application Publication
(10) Pub. No.: US 2009/0291121 A1
(43) Pub. Date: Nov. 26, 2009

(54) CAPSULE AND COATED CAPSULES AS A DELIVERY SYSTEM FOR DIETARY SUPPLEMENTS AND THERAPEUTIC MATERIALS

(75) Inventors: Hartmut HAAS, Bevern (DE); Thomas MANGOS, Fort Lee, NJ (US); Donna ROSA, Nutley, NJ (US); Martin GURNEY, East Hanover, NJ (US); Deborah KENNISON, Wayne, NJ (US)

Correspondence Address: Gregory A. Nelson;Novak Druce & Quigg LLP, 525 Okeechobee Blvd, Suite 1500, West Palm Beach, FL, 33401, US

(73) Assignee: Symrise, Inc., Teterboro, NJ (US)

(21) Appl. No.: 12/472,048

(22) Filed: May 26, 2009

Related U.S. Application Data
(60) Provisional application No. 61/055,726, filed on May 23, 2008.

(51) Int. Cl. A61K 9/48; A61K 31/485; A61K 31/164; A61K 31/355; A61P 43/00

(52) U.S. Cl. 424/439; 514/289; 514/626; 514/458

(57) ABSTRACT

A coated spherical capsule that comprises a seamless capsule and an edible coating is disclosed. The seamless capsule includes a seamless solid shell surrounding a mononuclear liquid or viscous core and the edible coating surrounding the seamless capsule. The edible coating can include at least one dietary supplement and at least one sugar or sugar alcohol. The liquid or viscous core can include 1-100% of a flavoring, based on total weight of the liquid or viscous core. The edible coating can include an effective amount of the at least one dietary supplement based on the weight of the edible coating. Also disclosed is a method of administering an effective amount of a dietary supplement. The method includes providing a plurality of coated spherical capsule and ingesting at least one coated spherical capsule for two or more days in order to deliver an effective amount of a dietary supplement.

### CROSS REFERENCE TO RELATED APPLICATIONS

This application claims priority to U.S. Patent Application Ser. No. 61/055,726, entitled “CAPSULE AND COATED CAPSULES AS A DELIVERY SYSTEM FOR NUTRACEUTICALS AND THERAPEUTIC MATERIALS,” filed May 23, 2008, the entirety of which is incorporated herein by reference.

### FIELD OF THE INVENTION

A coated spherical seamless filled capsule containing nutraceuticals and therapeutic materials and the use thereof as a delivery system for nutraceuticals and therapeutic materials.

### BACKGROUND

Under physical strain, e.g., participation in athletic activities, the body requires minerals (electrolytes) and other relevant dietary supplements and actives that may be provided to the body during or after the activity for preservation and/or regeneration of bodily performance. Generally, mineral loss is compensated by drinking isotonic beverages. However, by definition, the concentration of active minerals in an isotonic mineral beverage is equal the concentration of the actives in the bodily fluids. Such mineral drinks have the disadvantage that they are not easily to handle during workout, e.g., they are heavy to carry while running. On the other hand, it is often undesirable to consume the large amounts of an isotonic beverage necessary to deliver the quantity of minerals lost as a function of sweating.

Coated chewing gum products are also a well known means of delivering the minerals lost during athletic activities. In these coated chewing gums, the coating may include low levels of calcium carbonate as an anti-caking agent. However, the levels present in coated chewing gums are not high enough to be used as a dietary supplement or antacid. For example, U.S. Pat. No. 4,867,989 discloses a chewing gum composition coated with an outer shell containing layers of a mineral compound and a coating syrup.

Antacids are usually taken on an “as needed” basis to relieve gastrointestinal disturbances mostly due to dietary indiscretions. Antacids are generally inorganic mineral salts such as calcium carbonate, magnesium carbonate, calcium hydroxide, magnesium hydroxide, aluminum hydroxide or bismuth subsalicylate. Antacids readily neutralize acids in the gastrointestinal (GI) tract and are commonly available as antacid tablets or liquids.

Previously, antacids have been added to a chewing gum matrix or in a chewing gum coating. However, such products have not been totally acceptable to consumers. The large amount of active antacid needed for effectiveness does not lend itself to giving a good tasting product chewing gum delivery system. In addition, when added as a filler in the chewing gum matrix, the antacid is entrapped in the chewing gum matrix and is not released upon chewing.

### SUMMARY OF THE INVENTION

In one embodiment, the invention is drawn to a coated spherical capsule that includes a seamless capsule, comprising a seamless solid shell surrounding a mononuclear liquid or viscous core; and an edible coating surrounding the seamless capsule. The edible coating can include at least one dietary supplement and at least one sugar or sugar alcohol. The liquid or viscous core can include 1-100 wt-% of a flavoring, based on total weight of the liquid or viscous core. The edible coating can include 5-70 wt-% of the at least one dietary supplement based on a weight of the edible coating. The edible coating comprises 15-40 wt-% of the at least one dietary supplement based on the weight of the edible coating.

The edible coating can include an effective amount of at least one bulk mineral, at least one trace mineral, or both. The coated spherical capsule also include at least one oil soluble compound that enhances absorption of the at least one mineral, the at least one trace mineral, or both. The edible coating can include an effective amount of at least one bulk mineral selected from the group consisting of calcium, zinc, iron, magnesium, potassium, phosphorous, sodium, ions thereof, and combinations thereof.

The liquid or viscous core comprises at least one oil soluble vitamin or pro-vitamin. The at least one oil soluble vitamin or pro-vitamin can be selected from the group consisting of Vitamin E, Vitamin K, Vitamin A, beta-carotene, carotenoids, and combinations thereof.

The edible coating comprises at least one water-soluble vitamin. The water-soluble vitamin is selected from the group consisting of Vitamin B, Vitamin C, and combinations thereof. The edible coating can include a mixture of (i) Zinc with Vitamin C, (ii) Calcium with Vitamin D, (iii) Selenium with Vitamin B12, or (iv) a combination thereof.

The coated spherical capsule can include at least 20% of the Dietary Reference Intake (RDI) or the Recommended Dietary Allowance (RDA) of at least one dietary supplement.

The coated spherical capsule can include at least one botanical extract. The at least one botanical extract can be selected from the group consisting of Echinacea, St John's Wort, Ginseng, Tea polyphenols, extracts thereof, and combinations thereon.

The coated spherical capsule can include at least one therapeutic agent for: (i) treating acid reflux disease or symptoms thereof, (ii) providing an anti-inflammatory effects, (iii) providing an antiseptic effect, (iv) providing an analgesic effect, or (v) a combination thereof. The at least one therapeutic agent can be selected from the group consisting of salts of bismuth, magnesium, aluminum, calcium, and combinations thereof.

The coating can comprise at least two layers comprising different dietary supplements, different compounds, or both. The selected different dietary supplements can have properties such that they will react if exposed to one another. The at least two layer can be separated by a third layer that prevents the different dietary supplements from reacting with one another in coated spherical capsule form.

Also disclosed is a method of administering an effective amount of a dietary supplement. The method can include providing a plurality of coated spherical capsule and ingesting a quantity of the coated spherical capsules on at least two consecutive days in order to deliver an effective amount of at least one dietary supplement present in the coated spherical capsules. The at least one dietary supplement can be a bulk mineral selected from the group consisting of calcium, zinc, iron, magnesium, potassium, phosphorous, sodium, ions thereof, and combinations thereof. The ingesting step can include ingesting a quantity of said coated spherical capsules on at least two consecutive days in order to deliver at least 50% of a Dietary Reference Intake (RDI), a Recommended Daily Allowance (RDA), or both, of at least one dietary supplement present in the coated spherical capsules.

These and other features, objects and advantages of the present invention will become more apparent to one skilled in the art from the following description.

### DETAILED DESCRIPTION

The presented invention describes a flavored confectionery-type product intended for ingestion and concomitant administration of dietary supplements useful for improving health and fitness. As used herein, “dietary supplement” refers to a composition that supplements nutrients, including vitamins, mineral, fatty acids, and amino acids. As will be recognized, some minerals and mineral salts can be used as antacids. Thus, such antacids will be encompassed by the term dietary supplement.

In one embodiment, a coated spherical capsule is disclosed that includes a seamless capsule, comprising a seamless solid shell surrounding a mononuclear liquid or viscous core; and an edible coating surrounding the seamless capsule. The edible coating can include at least one dietary supplement and at least one sugar or sugar alcohol. The liquid or viscous core comprises 1-100 wt-% of a flavoring, based on total weight of the liquid or viscous core, and the edible coating can include 5-70 wt-% of the at least one dietary supplement based on the weight of the edible coating.

The liquid or viscous core can include at least 3 wt-% of a flavoring based on the total weight of the liquid or viscous core, or at least 5 wt-% of a flavoring, or at least 10 wt-% of a flavoring. The edible coating can include 10-60 wt-% of at least one dietary supplement based on the weight of the edible coating, or at least 15-40 wt-% of the at least one dietary supplement.

The edible coating material can include an effective amount of at least one bulk mineral, at least one trace mineral, or both. As used herein, an “effective amount” of a dietary supplement is at least 10% of the Dietary Reference Intake (RDI) or the Recommended Dietary Allowance (RDA) of at least one dietary supplement, or 10% of amount otherwise determined to produce health or wellness benefits. For example, the current RDI for calcium is 1000 mg, so an effective amount of calcium would be 100 mg.

The coated spherical capsule further includes at least one oil soluble compound that enhances absorption of at least one mineral, at least one trace mineral, or both.

The liquid or viscous core can also include at least one oil soluble vitamin or pro-vitamin. The at least one oil soluble vitamin or pro-vitamin can be selected from Vitamin E, Vitamin K, Vitamin A, beta-carotene, carotenoids, and combinations thereof.

The edible coating can include an effective amount of at least one bulk mineral selected from the group consisting of calcium, zinc, iron, magnesium, potassium, phosphorous, sodium, ions thereof, and combinations thereof. The edible coating can include at least one water-soluble vitamin. The water-soluble vitamin can be selected from the group consisting of Vitamin B, Vitamin C, and combinations thereof.

The edible coating can include a mixture of Zinc with Vitamin C, Calcium with Vitamin D, Selenium with Vitamin B12, or a combination thereof.

The coated spherical capsule comprises at least 20% of the Dietary Reference Intake (RDI) or the Recommended Dietary Allowance (RDA) of at least one dietary supplement. The coated spherical capsule can include at least 40% of the Dietary Reference Intake (RDI) or the Recommended Dietary Allowance (RDA) of at least one dietary supplement, or at least 50% of at least one dietary supplement, or at least 66% of at least one dietary supplement, or at least 100% of at least one dietary supplement.

The coated spherical capsule can include any of the preceding relative amounts of the DR1 or RDA value for at least two, at least three, at least four, or at least five dietary supplements. Similarly, the coated spherical capsules can include any combination of the preceding relative amounts for a variety of different dietary supplements. For example, a coated spherical capsule can include at least 20% of the RDA for calcium, potassium and sodium, and at least 66% of the RDA for Magnesium.

The coated spherical capsule can include at least one botanical extract. The at least one botanical extract can be a botanical extract selected from the group consisting of Echinacea, St John's Wort, Ginseng, Tea polyphenols, extracts thereof, and combinations thereon.

The coated spherical capsule can include at least one therapeutic agent for: (i) treating acid reflux disease or symptoms thereof, (ii) providing an anti-inflammatory effects, (iii) providing an antiseptic effect, (iv) providing an analgesic effect, or (v) a combination thereof. The at least one therapeutic agent can be a therapeutic agent selected from the group consisting of salts of bismuth, magnesium, aluminum, calcium, and combinations thereof.

The edible coating can include at least two layers containing different dietary supplements, different compounds, or both. The edible coating can include at least two layers comprising different dietary supplements, wherein the different dietary supplements will react with one another. The at least two layer containing the different dietary supplements can be separated by a third layer preventing the different dietary supplements from reacting with one another prior to ingestion.

In another embodiment, the invention is drawn to a method of administering an effective amount of a dietary supplement. The method includes providing a plurality of coated spherical capsule containing a dietary supplement and ingesting a quantity of the coated spherical capsules on at least two consecutive days in order to deliver an effective amount of at least one dietary supplement present in the coated spherical capsules. The ingesting step can include ingesting the coated spherical capsules on at least seven consecutive days, on at least fourteen consecutive days or on at least five of seven consecutive days.

The ingesting step can include ingesting a quantity of the coated spherical capsules on at least two consecutive days in order to deliver at least 50% of a Dietary Reference Intake (RDI), a Recommended Daily Allowance (RDA), or both, of at least one dietary supplement present in the coated spherical capsules. Similarly, the ingesting step can result in delivery of at least 75% or at least 100% of the Dietary Reference Intake (RDI), a Recommended Daily Allowance (RDA), or both, of at least one dietary supplement present in the coated spherical capsules.

The primary objective of the invention is to provide the consumer with a pleasant delivery system for nutraceuticals such as minerals in a convenient form suitable to provide the body with required quantities of minerals related to Dietary Reference Intake (RDI), Adequate Intake (AI), Tolerable Upper Intake Levels (UL), and Recommended Daily Allowance (RDA), without being dependent on isotonic beverages or pressed tablets. An effective flavored delivery system is disclosed to promote a convenient and pleasant way for intake of necessary quantities of minerals when needed.

The confectionery-type form of product disclosed is a seamless capsule consisting of a thin shell and a large liquid core filled with an effective amount of a flavor. The seamless capsule is coated with an effective amount of the nutraceutical, other ingredients of interest and mixtures thereof. The coating is applied using a proper formulation added via a pan coating or other suitable processes such as a coating turbine or a tumbler or the like to produce a functional dragée or chewy dragée, the term used to resemble sugar- or sugar-free type coated confection.

Such coated seamless capsules can serve as a vehicle for the administration of mineral and non-mineral nutraceuticals and other active agents to promote fitness and other health aspects in a pleasant way. In particular, by providing a pleasant tasting method for the delivery of vitamins, botanical extracts, and medicaments such as antacids, and combinations thereof. The active agents can be included in a coating of the coated seamless capsule as part of a water soluble matrix, such that once placed in the mouth, the active agent in the matrix is released quickly. This allows a coating to be a carrier for an active agent, such as an antacid, having fast release characteristics and a pleasant taste.

Although microcapsules have been used for breath freshening and breath protection agents before, many actives are not soluble in the core of the capsule. It has been found that by incorporating certain actives in the shell of the inner capsule, in the protecting layer or in the coating, these problems of insolubility associated with other microcapsules can be avoided. Additionally, development of suspensions and dispersions of oil-insoluble actives in the inner core liquid via homogenization techniques and control of viscosity have been established to achieve long-term stability of immiscible two phase systems in the core.

On the other hand, some oil-soluble supplement may be better delivered in the liquid core of the inner capsule via solubilization in the lipophilic solvent system to achieve an acceptable daily intake, since they are not compatible with the coating process. It is therefore an objective of the present invention to provide improved methods of delivery for minerals, vitamins and flavor of variable physical properties and solubility and mixtures thereof at effective levels, which cannot be achieved with a capsule system alone or a coated confectionery product.

Achieving high process stability of the inside capsule coupled with rapid solubility of the capsule shell in the mouth is a particular technical problem. On the one hand, it is advantageous, specifically for the process for preparation of the capsule and for storage, to select a shell formulation that gives the coating-free capsule a particularly high mechanical strength and rapidly forms a solid gel that after drying is as hard as possible and absorbs little water. On the other hand, for a good sensation in the mouth on consumption of the final coated capsule it is advantageous if the shell absorbs water rapidly, is soft and flexible and dissolves quickly.

The diameter of the coated seamless capsules of the invention can range from 3 to 30 mm, of 4 to 20 mm or even 5 to 9 mm. Thus, on consumption of the coated capsules, a relatively large amount of core liquid passes directly into the mouth. This provides an immediate flavor impression or flavor burst. The impact of the flavor impression can be adjusted by modifying the type and/or amount of flavoring in the core liquid. The unpleasant flavor notes and other effects, such as astringency, are reduced or eliminated because of the rapid release of the flavored core.

Mixtures of flavorings with vegetable oils or triglycerides are preferably used for the core liquid. Examples of suitable flavorings are synthetic and natural flavorings and mixtures thereof as well as also essential oils, oleoresins or extracts of plants, leaves, flowers, fruit and the like, as well as combinations thereof. Flavorings from the series comprising but not limited to peppennint oil, spearmint oil, eucalyptus oil, cinnamon oil, cassia oil, aniseed oil, bitter almond oil, oil of cloves, parsley seed oil, citrus oils, fruity flavoring compositions having tastes oriented towards, for example, apple, pear, peach, grape, strawberry, raspberry, cherry, pineapple and combinations thereof, are preferably used.

For reference purposes, types of ingestible seamless filled capsules, preferentially based on tailored gelatin mixtures or mixtures of gellan and gelatin, suitable for coatings and a process for applying the coating to seamless filled capsules is given in US 2006/0110442 A1 by Wonschik et al., the entirety of which is incorporated herein by reference. The method describes the use of sugar-containing or sugarless coating syrup as the coating of the seamless filled capsules, which may comprise an intermediate layer or intermediate layers arranged between the capsule shell and the coating, for improvement of adhesion between the shell and the coating. In addition to water and sugar or sugar alcohols, the coating solution may include up to 5% of optional constituents including high intensity or bulk sweeter, food acids, flavor, and color. The inner or coated capsules may be additionally protected by a layer of wax or other materials for visual appeal and/or functionality

The invention describes the use of suspensions of micronized or non-micronized minerals and other inorganic salts, vitamins and other actives to the sugar-containing or sugarless coating syrup to thus be included in the coating of the seamless filled capsules. The coating enclosing the seamless filled capsules can contain any macrominerals and trace minerals and desirable mixtures thereof as dietary supplement for specific health benefits, activities, age groups, gender, release characteristics, taste, etc, be it in a water-soluble form for dissolution in the coating syrup or a water-insoluble form for dispersion in the coating syrup or center capsule.

### Macro Minerals

A variety of elements are required to support the biochemical processes, many play a role as electrolytes or in a structural role. In human nutrition, the dietary bulk minerals have a recommended daily allowance (RDA) of greater than 200 mg/day. As used herein, “bulk minerals” include the mineral in both mineral form, i.e., neutral, and ionic form. Exemplary bulk minerals include, but are not limited to:

Calcium in the form of calcium carbonate, calcium gluconate, and other salts, for contributing to muscle health, digestive system health, cardiovascular health, building bones, neutralizing acidity and eliminating toxins;

Chloride for production of hydrochloric acid in the stomach;

Magnesium for processing adenosine triphosphate (ATP) and facilitating related reactions that contribute to health, building bone, producing strong peristalsis, increasing flexibility, and increasing alkalinity;

Phosphorus for bone building and mineralization, e.g., apatite, energy processing, and contributing to overall health;

Potassium for contributing to cardiovascular health and nerve health, and serving as an electrolyte;

Sodium, including sodium chloride, for contributing to overall health and serving as an electrolyte and, in combination with chlorine, regulating fluid movement and minerals in and out of body cells; and

Sulfur for three essential amino acids and many proteins and cofactors related to skin, hair, nails, liver, and pancreas health.

### Trace Minerals

A variety of elements are required in trace amounts, usually because they play a role in catalysis in enzymes. Trace mineral elements have a recommended daily allowance less than 200 mg/day. As used herein, “trace minerals” include the mineral in both mineral form, i.e., neutral, and ionic form. Exemplary trace mineral elements include, but are not limited to:

Cobalt, which is required for biosynthesis of the vitamin B12 family of coenzymes;

Copper, which is a required component of many redox enzymes, including cytochrome c oxidase;

Fluorine strengthens tooth enamel which contains fluoroapatite;

Iodine, which is required for the biosynthesis of thyroxin;

Iron, which is required for many proteins and enzymes, notably hemoglobin;

Manganese, which contributes to processing of oxygen;

Molybdenum, which is required for xanthine oxidase and related oxidases;

Nickel, which is present in urease;

Selenium, which is required for the activity of antioxidant proteins including peroxidases;

Vanadium, which is believed to be important to overall health; and

Zinc, which is required for several enzymes such as carboxypeptidase, liver alcohol dehydrogenase, carbonic anhydrase.

It should be noted that iodine is required in larger quantities than the other trace minerals and is sometimes classified with the bulk minerals or macrominerals. Sodium is not generally found in dietary supplements, despite being needed in large quantities, because the ion is very common in food. Many other elements have been suggested as required in human nutrition, in varying quantities. However, not all have been definitively established as essential to human nutrition. Exemplary elements for which convincing scientific evidence is lacking include, but are not limited to: Boron, Bromine, Chromium, Indium, Rubidium, Silicon, and Titanium. It is to be understood that the coated microcapsules disclosed herein can be adapted for delivery of any number of bulk minerals, trace minerals, and other minerals.

It is desired that the coated capsules disclosed herein are suitable for immediate consumption without further processing and can be sucked or chewed in the mouth without the shell being found to be annoying, in particular without being sticky or tacky, i.e., without sticking to teeth, tongue, gums or palatine. In addition a pleasant mouth-feel and flavor should be experienced when consuming the spherical capsule even though a large amount of nutraceutical may be consumed.

The coated capsules can include a coating-free spherical seamless capsule having a shell only 20-200 μm thick, a diameter in the range of 3-7 mm and a shell thickness to capsule diameter ratio in the range of 0.005-0.05. The shell which is still moist during the preparation prior to drying remains dimensionally stable even at temperatures of 40° C.-60° C. By this means both a higher drying rate (as a result of the possible use of higher drying temperatures) and also improved storability and transportability were to be achieved.

The spherical coated capsule can include:

(a) a coating-free capsule having (i) a liquid or viscous core and (ii) a seamless solid shell surrounding this core, and

(b) a seamless, solid coating surrounding said coating-free capsule, wherein

the diameter of the coated capsule can be in the range of 5-9 mm,

the solid coating comprises at least one sugar or sugar-alcohol in an amount from about 30-90% (m/m), based on the total mass of the coated capsule,

the diameter of the coating-free capsule is in the range of 3-7 mm,

the thickness of the shell of said coating-free capsule is in the range of 20-200 μm,

the ratio of shell thickness to diameter of said coating-free capsule is in the range of 0.004-0.04,

the shell of said coating-free capsule contains 70-90% (m/m) gelatin or alginate and 10-30% (m/m) plasticizer, based on the solids content of said shell, and

the core has a flavoring content in the range of 1-100% (m/m), based on the total mass of the core.

The coating-free capsule of the capsules of the present invention can be prepared by a method comprising the following steps:

pumping a liquid or viscous core material and a gelatin or alginate-containing curable shell mixture simultaneously through a concentric multi-component nozzle so that they drip into a cooling liquid with the formation of a capsule,

drying said capsule, and

coating the resulting dried capsule (hereinafter also: coating-free capsule), optionally only after applying an intermediate layer or intermediate layers to the dried capsule.

Throughout the present description a capsule is designated a spherical capsule insofar as the ratio between the largest and the smallest diameter of the capsule is not more than 1.2. This arithmetic mean of the largest and the smallest diameter of the coating-free capsule is designated as the diameter of a coating-free capsule according to the invention below.

For the organoleptic assessment and flavor impact, it is particularly advantageous if the diameter of the coating-free capsule is in the range of about 3-6 mm, the thickness of the shell is in the range of about 50-150 μm and the ratio of shell thickness to coating-free capsule diameter is in the range of about 0.01-0.03. It is most advantageous if the diameter of the coating-free capsule is in the range of 4-6 mm, the thickness of the shell is in the range of 50-90 μm and the ratio of shell thickness to diameter of the coating-free capsule is in the range of 0.01-0.02.

Unless indicated otherwise, all percentages and ratios given are by weight and all conditions (e.g. state of aggregation) refer to 20° C.

### The Coating

The coating mixture is based on solid sugars or sugar alcohols. The solid sugars or sugar alcohols typically are in crystalline form. Sugar alcohols are preferred, particularly for oral care/oral hygiene capsules, for example in breath freshening applications. The diameter of the coated capsule of the present invention, i.e., after coating the coating-free capsule, is in the range of 3-30 mm. The coating mixture will normally be applied to the coating-free capsule in aqueous form. Typically the sugars or sugar alcohols are used in the coating process as aqueous solutions.

The coating mixture for use in the preparation process according to the present invention can comprise or consist of 15 to 45% of water, 50 to 85% of sugar(s) or sugar alcohol(s), with the remainder being optional other constituents. The coating mixture can comprise or consist of 20 to 40% of water, 60 to 80% of sugar, 5 to 35% of functional or nutraceutical actives, and 0 to 5% of optional constituents.

The coating mixture preferably comprises one or more sugar alcohols, preferably selected from the group consisting of erythritol, isomalt, lactitol, maltitol, mannitol, sorbitol, xylitol and hydrogenated starch hydrolysates, isomalt, mannitol and xylitol being preferred.

The coating of the coated capsule according to the present invention comprises one or more sugar(s) or sugar alcohol(s) in an amount from about 30-90%, or 40-85%, or even of 55-80%, based on the total mass of the coated capsule. The residual amount of water in the coating after the coating process typically is less than 2%, or less than 1%, based on the weight of the coating of the coated capsule.

The coating mixture can be applied to the coating-free capsule via panning or dragee coating processes, involving dragee pan, coating turbine, tumbler or the like. During the coating process, the coating is applied to the coating-free capsule in several layers. The coating can include 5 to 300 layers, or even 10 to 50 layers. The number of layers can be widely varied within the mass limits of sugar(s) or sugar alcohol(s) given above, depending on the capsule properties to be achieved. The shell of preferred coating-free capsules may dissolve in the mouth in less than 60 seconds, or a thicker coating can be applied for slow dissolution. The layers can contain both (i) functional or nutraceutical actives and (ii) sugar(s) or sugar alcohol(s).

### Actives in the Coating

Minerals and salts of calcium, salts of inorganic or organic ingredients, antacids and/or other functional or nutraceutical actives are either added directly to the coating mixture or by partially replacing sugar(s) or sugar alcohols not to exceed the maximum solid content of the coating mixture.

The preferred calcium salt is calcium carbonate, but other salts and minerals could be used that contain calcium, including, but not limited to, calcium ascorbate, calcium acetate, calcium bisglycinate, calcium carbonate, calcium chloride, calcium chloride dehydrate, calcium chloride hexahydrate, calcium citrate, calcium citrate malate, calcium citrate tetrahydrate, calcium famarate, calcium glubionate, calcium glubionate monohydrate, calcium gluceptate, calcium gluconate, calcium gluconate monohydrate, calcium glutarate, calcium glycerophosphate, calcium hydrolyzed animal protein (HAP) chelate, calcium (hydrolyzed vegetable protein (HVP) chelate, calcium hydroxide, calcium lactate, calcium lactate gluconate, calcium lactate pentahydrate, calcium lactate trihydrate, calcium lactobionate dehydrate, calcium levulinate, calcium levulinate dehydrate, calcium malate, calcium oxide, calcium phosphate dibasic, calcium phosphate monobasic, calcium phosphate tribasic, calcium pidolate, calcium pyrophosphate, calcium silicate, calcium sodium lactate, calcium succinate, calcium sulfate, calcium sulfate dehydrate, hydroxylapatite and derivatives thereof where the OH moiety is replaced by fluoride, chloride or carbonate, coral, dolomite, bone meal, oyster shell, egg shell, and mixtures thereof.

The coating mixture for use in the preparation process according to the present invention preferably comprises or consists of 15 to 45% of water, preferably from 20 to 80% of sugar(s) or sugar alcohol(s), and 1 to 40% of functional or nutraceutical actives. The coating mixture can include 3 to 35% functional or nutraceutical active, 10 to 30% functional or nutraceutical actives or 15 to 25% functional or nutraceutical actives.

The ratio of functional or nutraceutical actives to sugar(s) or sugar alcohol(s) in the coating can range from 1:0.5 to 1:80, or from 1:0.5 to 1:40, or from 1:0.5 to 1:35, or from 1:0.5 to 1:1.4, or from 1:0.5 to 1:1.

Formulations according to the invention can advantageously also comprise minerals and salts other than of calcium. For example, the formulations can contain magnesium, potassium, sodium, phosphorus, chloride, zinc, manganese, iron, cobalt, copper, boron, chromium, manganese, molybdenum, nickel, selenium silicon, tin, vanadium, their corresponding salts and chelates, and combinations thereof. The preceding list is not intended to be exhaustive.

Formulations according to the invention can advantageously also comprise minerals and salts thereof to control acid reflux. Minerals and salts thereof that are useful for treating and controlling acid reflux include, but are not limited to, calcium carbonate, magnesium carbonate, calcium hydroxide, magnesium hydroxide, aluminum hydroxide and bismuth salts, such as bismuth sub-salicylate, and combinations thereof.

### Additives in the Coating (Optional):

High-intensity sweeteners can also be added to the coating according to the invention. In order to provide improved adsorption of calcium, the coating may also include Vitamin D, e.g., vitamin D2 (Ergocalciferol) or vitamin D3 (Cholecalciferol). Variable ratios of calcium and vitamin D compounds can be utilized in order to meet with Recommended Daily Allowance (RDA) and Adequate Intake (AI) standards. Similarly, the ratios of nutraceutical actives can be adjusted to help in the development and maintenance of bones and teeth, help in the adsorption and use of calcium and phosphorous, help to prevent vitamin D deficiency or reduce the risk of developing osteoporosis.

The nutraceutical active in the formulations according to the invention can advantageously include vitamins and vitamin precursors other than provitamin D, vitamin D (calciol) and its derivatives (e.g. vitamin D2, vitamin D3), which are suitable food uses. Vitamins and vitamin precursors which may be mentioned by way of example include, but are not limited to, vitamin A (retinol) and its derivatives, vitamin B1 (thiamine) and its salts, vitamin B12 (cobalamin), vitamin B2 (riboflavin) and its derivatives, vitamin B3 and its derivatives, vitamin B4 (adenine) and its derivatives, provitamin B5, vitamin B5 (pantothenic acid) and its derivatives, vitamin B6 (pyridoxol, pyroxidal, pyridoxamine) and its derivatives, vitamin C (ascorbic acid) and its derivatives, vitamin E (D-alpha-tocopherol) and its derivatives, vitamin F (essential fatty acids, linolenic acid and linoleic acid) and its derivatives (e.g. vitamin F ethyl ester, vitamin F glyceryl ester), vitamin H (vitamin B7, biotin), vitamin KI (phylloquinone, phytonadione) and vitamin K3 (menadione, menaquinone).

### Optional Constituents of the Coating

To improve the adhesion or bonding of the coating to the coating-free capsule, the surface of the coating-free capsule can be roughened or auxiliary materials can be applied to the coating-free capsule before the coating process with the nutraceutical actives and sugar or sugar alcohol based coating materials is carried out. Roughening of the surface of the coating-free capsule can be achieved by adding corn starch or a similar adjuvant in powder form to the wet coating-free capsule before the drying step.

Suitable auxiliary materials that can also be used include, but are not limited to, gum Arabic, maltodextrin, starch, sugar, sugar alcohol, gelatin, and combinations thereof. These auxiliary materials can be applied using an aqueous solution containing about 40 to about 60% of the auxiliary materials. The coating-free capsule can be coated with one ore two layers of auxiliary material before performing the actual coating process with the sugar or sugar alcohol based coating materials. Colorants or dyes that may be included in the coating are water-soluble food colorants. Exemplary colorants include, but are not limited to, FD&C Blue #5, FD&C Yellow #1, brilliant blue, allura red, inorganic pigments, like edible metal oxides, e.g. titanium dioxide, and combinations thereof.

In addition, during application of the auxiliary material to the coating-free capsule, it is possible to add actives to the aqueous auxiliary material solution. For example, suitable actives are the breath control actives, antitussive actives or oral anesthetic actives given above. If desired, flavorings, like those mentioned above as constituent of the liquid or viscous core, can be added to the coating mixture.

Solid acids can be added to the coating mixture. Exemplary solid acids include, but are not limited to, the monoacid, diacid or triacid type, preferably citric acid, fumaric acid, malic acid, or lactic acid. The use of at least one such acid makes it possible, to tailor its sensory properties. Typically the amount of acid(s) added to the coating mixture is from about 0.5 to about 1.5%.

If a shiny final coating is preferred, waxes or shellac can be applied to the coated capsules, usually one or two layers of wax or shellac are sufficient. In addition the shiny final coating can prevent the coated capsules from sticking to each other and from sticking to the hands of the consumer. Waxes, like bees wax, carnauba wax or paraffins are applied in pure form. Shellac can be applied using 3 to 10% in an aqueous solution.

### Appearance

Capsules according to the invention can be ballshaped, e.g., spherical. The ratio between the largest and the smallest diameter of a spherical coated capsule according to the invention is not more than 1.2, preferably not more than 1.1.

### Flavoring Content in the Liquid or Viscous Core

The core liquid in the coating-free capsule can be liquid or viscous, up to paste-like. On consumption of the coated capsules according to the invention a relatively large amount of core liquid passes directly into the mouth. This gives rise to an immediate flavor impression, the impact of the flavor impression can be adjusted by type and/or amount of flavoring in the core liquid.

Mixtures of flavorings with vegetable oils or triglycerides can be used for the core liquid. The flavoring mixture is preferably a clear solution at room temperature and preferably also a clear solution at 10 degrees Celsius. Examples of suitable flavorings are synthetic and natural flavorings and mixtures thereof as well as oleoresins or extracts of plants, leaves, flowers, fruit and the like, as well as combinations thereof. Flavorings from the series comprising peppermint oil, spearmint oil, eucalyptus oil, cinnamon oil, cassia oil, aniseed oil, bitter almond oil, oil of cloves, parsley seed oil, citrus oils, fruity flavoring compositions having tastes oriented towards, for example, apple, pear, peach, grape, strawberry, raspberry, cherry, pineapple and combinations thereof, are preferably used.

Additional individual flavor substances that can be used as part of the core flavoring mixture include those having a cooling or refreshing effect in the throat or the oral or nasal cavity. Exemplary flavor substances exhibiting a cooling or refreshing effect include, but are not limited to, menthol, menthone, carboxamides, menthol acetate, menthol methyl ether, methone acetals, menthol carbonates, menthol succinates, 1,s-cineol (eucalyptol), carvone, alpha-terpineol, thymol, methyl salicylate, 2′-hydroxypropiophenone.

### Flavoring Content in the Liquid or Viscous Core

The flavoring content in the core liquid depends, in particular, on the capsule size and the flavoring intensity and according to the invention ranges from 1-100%, based on the total mass of the liquid or viscous core. However, a flavoring content in the liquid or viscous core in the range of 5-90%, or 30-80%, based on the total mass of the liquid or viscous core.

### Sweeteners in the Liquid or Viscous Core (Optional)

Sweeteners can also be added to the core liquid of a capsule according to the invention, with the use of solubilizing agents if appropriate. Since, in accordance with the intention, the core liquid comes into direct contact with the teeth in the mouth, it is advantageous if the core liquid does not exert a pH-lowering action.

While developing the coated capsules disclosed herein, it was determined that thaumatin, neohesperidine, miraculin, and mixtures thereof, are particularly suitable as sweeteners in the core liquid. It was determined that these sweeteners do not have an adverse effect on the pH value. Thaumatin is particularly preferred.

It was also determined that other sweeteners that are commonly used, such as, for example, saccharinic acid or acesulfame K lower the pH value of the aqueous phase. Thus, the coated capsules disclosed herein can be free of saccharinic acid or acesulfame K.

### Oils in the Liquid or Viscous Core

Oils useful herein include triglycerides, and especially triglycerides of caprylic acid and capric acid, mixtures of triglycerides of the vegetable oil type, olive oil, sunflower oil, corn oil, peanut oil, grape seed oil, wheat germ oil, mineral oils and silicone oils. Preferred oils for diluting the flavorings used are, in particular fractionated coconut oils which contain mainly C₆-C₈ fatty acid radicals. These oils are characterized by their neutral taste and by their good stability to oxidation.

### Actives in the Liquid or Viscous Core (Optional)

Coloring substances, vitamins (e.g. ascorbic acid, vitamin E), an/or vegetable extracts can be added to the core liquid.

Antitussive actives can be added to the core. Exemplary antitussive actives include, but are not limited to, dextromethorphan, chlophedianol, carbetapentane, caramiphen, nosciapine, diphenylhydramine, codeine, hydrocodone, hydromorphone, fominoben, benzonatate, and combinations thereof.

Oral anesthetic actives can be added and include e.g. phenol, lidocaine, dyclonine, benzocaine, menthol, salicyl alcohol and hexylresorcinol.

The core of the inner capsule can furthermore and advantageously comprise oil-soluble vitamins and vitamin precursors, which are suitable for use in food. Vitamins and vitamin precursors which may be mentioned by way of example are: vitamin A (retinol) and its derivatives, provitamin D, vitamin D (calciol) and its derivatives (e.g. vitamin D2, vitamin D3), vitamin E (D-alpha-tocopherol) and its derivatives, vitamin F (essential fatty acids, linolenic acid and linoleic acid) and its derivatives (e.g. vitamin F ethyl ester, vitamin F glyceryl ester), beta carotene, lutein, and other functional carotenoids.

### Further Constituents in the Liquid or Viscous Core (Optional)

The formulations according to the invention may comprise cooling agents. Preferred cooling active compounds for use in formulations according to the invention are listed in the following. The person skilled in the art can supplement the following list with a large number of further cooling active compounds; the cooling active compounds can also be employed in combination with one another. Preferably, the formulations according to the invention comprise at least one cooling active compound, preferably two or more cooling active compounds, chosen from the group consisting of:

Menthone glycerol acetal (trade name: Frescolat®, Symrise GmbH & Co. KG, Germany), menthyl lactate (trade name: Frescolat®ML, Symrise GmbH & Co. KG, Germany), substituted menthyl-3-carboxylic acid amides, 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, and mixtures thereof, i.e. Optacool® (Symrise, Germany), menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl 3,6-di- and -trioxaalkanoates, 3-menthyl methoxyacetate and icilin.

Formulations according to the invention that comprise l-menthol and at least one, particularly preferably at least two cooling substances are preferred according to the invention. Preferably, formulations according to the invention comprises a mixture of flavoring and/or aroma substances which imparts an overall herbal (herb-like), minty, cinnamon-like, clove-like, wintergreen and/or fruity character, to the formulation.

Preferred physiological warming agents for use in the coated capsules disclosed herein are those selected from the group consisting of vanillyl alcohol n-butyl ether, vanillyl alcohol, n-propylether, vanillyl alcohol, isopropyl ether, vanillyl alcohol isobutyl ether, vanillyl alcohol, n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, ethanol, iso-propyl alcohol, iso-amylalcohol, benzyl alcohol, chloroform, eugenol, cinnamon oil, cinnamic aldehyde, phosphate derivatives thereof, and mixtures thereof. The phosphate derivatives mentioned are those described in WO 97/102273. A commercial example of a suitable warming agent for use herein is Optaheat® (Symrise, Gennany). The warming composition can further comprise a cooling agent as described herein provided that the predominant effect is one of warming.

A salivating or moisturizing agent can be included in the preparation a coated capsule for use as a medicament for soothing irritated oral tissues. Exemplary salivating agents include, but are not limited to, trans-pelletorin or Optaflow® (Symrise, Germany), as described in U.S. Patent Application Publication No. 2004/0241312A and EP 1520850A2, or other actives, such as Jambu and Spilanthrol. These exemplary salivating agents modulate oral and nasal secretion, providing long-lasting moisturization of the oral and nasal tissue due to its lipophilicity by inducing salivation, in comparison with organic acids such as citric acid.

### Shell Thickness

For rapid dissolution of the shell of a coating-free capsule (as part of the capsule of the present invention) in the mouth the shell thickness should be as small as possible. For a constant core/shell mass ratio the shell thickness increases substantially with increasing diameter of the coating-free capsule.

The coating-free capsules have a shell thickness of only 20 μm-200 μm despite the large capsule diameter of 3-7 mm. In these capsules the ratio of shell thickness to capsule diameter is in the range of 0.004-0.04. These data relate to the dried capsule. The coating-free capsule diameter can be determined using a micrometer screw. For determination of the shell thickness a cross-section of the coating-free capsule is prepared. The thickness of the shell can be determined via a microscope with image processing. To this end the thickness of the coating-free capsule shell is measured at various points in the latter and the mathematical mean for the shell thickness is determined.

With the composition of the shell selected in accordance with the invention high process and transport stability, adequate elasticity, good biteability and a sufficiently high dissolving power in the mouth are ensured.

### Composition of the Shell:

### Viscosity of the Shell Mixture/Gel Point:

While not necessary for practicing the invention, in some embodiments the seamless capsule described herein can be formed using a multi-component nozzle. However, it must be understood that the invention is by no means limited to seamless capsules produced using a multi-component nozzle method and any limitations of the composition of the shell related to the multi-component nozzle method may or may not apply to shells made using alternate techniques.

When shaping a coating-free capsule described herein by means of a multi-component nozzle method particular attention has to be paid to the viscosity and the gelling characteristics of the curable shell mixture. Too low a viscosity or too low a gel point can prevent a capsule shell that is sufficiently solid in the wet state. Corresponding capsules would be mechanically destroyed by the further processing steps, such as, for example, centrifuging. Too high a viscosity and too high a gel point, on the other hand, prevent correct capsule shaping and, moreover, give rise to undesired, substantial satellite formation.

The viscosity of preferred shell mixtures for the preparation of coating-free capsules was determined using a CVO 120 rheometer (Bohlin Instruments GmbH, Pforzheim). The measurement system used was a plate-plate system with a plate diameter of 50 mm. The measurements were carried out using rotation. The shear rate was 50 s⁻⁵; the gap was set at 500 μm. The measurements were carded out isothermally; the temperature was 80° C.

At 80° C. preferred shell mixtures have a viscosity in the range of 30 mPas to 300 mPas, preferably of 40 mPas to 150 mPas, and particularly preferentially of 50 mPas to 90 mPas.

The gel point of preferred shell mixtures for the preparation of coating-free capsules was likewise determined using a CVO 120 rheometer (Bohlin Instruments GmbH, Pforzheim). The measurement system used was a plate-plate system with a plate diameter of 50 mm. The measurements were carried out using oscillation. The frequency was constant at 1 Hz, the gap was set at 500 μm and the temperature was lowered from 80° C. to 10° C. with a gradient of 5° C./min. The temperature at which the viscosity or memory module G′ is equal to the modulus of elasticity or loss module G″ was read off as the gel point, the sol/gel transition point (Thomas Mezger, Das Rheologie Handbuch, 2000).

The gel points of preferred shell mixtures are between 15° C. and 60° C., preferably between 20° C. and 40° C. and particularly preferentially between 25° C. and 35° C.

### Gelatin:

The shell of the coating-free capsules described herein contains gelatin or alginate and plasticizer, preference is given to gelatin. The grade and amount of the gelatin and of the plasticizer have an effect on the solubility kinetics of the shell in the mouth.

For shaping of the coating free capsule, aqueous solutions containing 10-40% (m/m), preferably 15-30% (m/m) and particularly preferentially 18-25% (m/m) gelatin are preferably used for the shell.

The gelatin used in the capsules according to the invention is in many cases obtained by partial hydrolysis of collagen-containing material from animals, such as, for example, pigs, cattle, fish or poultry. Type A gelatin is obtained by acid digestion, usually of pig or fish skins, whilst type B gelatin is obtained by alkaline digestion, usually of cattle bones and skins.

The term Bloom is used to characterize the gel strength of gelatin. In the determination of the Bloom value a stamp of a Bloom gelometer or texture analyzer 12.7 mm (0.5 inch) in diameter is pressed 4 mm deep into a 6.67% gelatin gel that has been aged for 18 hours at 10° C. prior to the measurement. The result is given in “Bloom”, corresponding to the weight in grams that is loaded onto the stamp in order to achieve the impression depth (see Schormuller, Handbuch der Lebensmittelchemie, Volume III, 1968 and British Standard Method for Sampling and Testing Gelatin (BS757; 1975)).

A gelatin having a Bloom value of more than 200, particularly preferentially having a Bloom value of 240-300, and specifically preferably a Type A gelatin is preferably used for the preparation of coating-free capsules according to the invention. By this means adequate stability of the shell is made possible during the preparation of the coating-free capsule and during transport, despite the small thickness of the capsule shell.

Gelatin grades that have been obtained from cattle, poultry or fish are also suitable for the preparation of the capsule according to the invention. In this context, in any event, as already mentioned, care must be taken that the viscosity and the gelling characteristics are correctly adjusted. Fish gelatins that can be used are both grades from cold water fish and grades from warm water fish. Mixtures of different gelatin grades can also be used.

Achieving high process stability of a coating-free capsule coupled with rapid solubility of the capsule shell in the mouth is a particular technical problem. On the one hand, it is advantageous, specifically for the process for preparation of the capsule and for storage, to select a shell formulation that gives the coating-free capsule a particularly high mechanical strength and rapidly forms a solid gel that after drying is as hard as possible and absorbs little water. On the other hand, for a good sensation in the mouth on consumption of the final coated capsule it is advantageous if the shell absorbs water rapidly, is soft and flexible and dissolves quickly.

Surprisingly, it has been found that this particular technical problem can be solved by the use of a mixture of a hydrolyzed gelatin with a Bloom value of 0 and a high-Bloom gelatin with a Bloom value of 200 and above (preferably a Bloom value in the range of 240-300). Presumably the high-Bloom gelatin forms a solid network here that is important for the process stability. The hydrolyzed 0-Bloom gelatin presumably occupies spaces in this network and in the mouth leads to a more rapid absorption of water and thus solubility of the entire shell.

Hydrolyzed 0-Bloom gelatin has no gelling power and is readily soluble in water at 20° C. In this 0-Bloom gelatin the polypeptide chains have been very substantially decomposed by acid or enzymatic hydrolysis. To date it is therefore also not used for the formation of coating-free capsule shells but is used only, for example, as a nutrient (protein source), as an emulsifier or also for clarifying wine.

Shell mixtures consisting of (a) hydrolyzed 0-Bloom gelatin, that has been obtained from any desired species of animal, with (b) gelatin that has a Bloom value of ≧200 are preferably suitable for solving the particular technical problem, the proportion of hydrolyzed 0-Bloom gelatin preferably being in the range of 0.5-90% (m/m), based on the solids content of the shell.

Here the gel point of the high-Bloom gelatin constituent is the decisive factor determining the gel point of the mixtures (see the appended table “Gel points”). The flexibility of a film produced from a shell mixture is, moreover, surprisingly high if the mixture contains O-Bloom gelatin.

A further possibility for solving the abovementioned particular technical problem consists in the mixture of certain low-Bloom (Bloom value <200) fish gelatins with high-Bloom gelatin (Bloom value ≧200).

The lower the Bloom value of a gelatin, the lower are, in general, the gel point, the viscosity and the mechanical stability of the moist solidified gel. Mixtures of gelatin having a low Bloom value and a medium Bloom value in order to achieve an improved solubility of the coating-free capsule shell in the mouth are known. U.S. Pat. No. 6,258,380 describes shells of this type. However, in this patent no specification of the gelatins has been given beyond the Bloom value.

Surprisingly, it has now been found that when fish gelatin is used as the gelatin fraction having a low Bloom value (<200) and at the same time a high-Bloom gelatin having a Bloom value of 200 and above is used, a further improved solubility of the coating-free capsule in the mouth can be achieved. This is presumably affected by the lower gel point of fish gelatins (below 28° C.) compared with pig, cattle and poultry gelatins (approximately 28-40° C.).

Fish gelatin grades with a gel point of <20° C. and grades that are prepared from cold water fish, for example cod, are particularly suitable since their gel points at approximately 10-20° C. are even below those of gelatins from warm water fish (gel point approximately 20-28° C.), such as, for example, from carp. In this context the cold water fish include all species of fish that live predominately in waters at temperatures of 18° C. and below. Research has confirmed the lower gel points of fish gelatins compared with pig, cattle and chicken gelatins with approximately comparable Bloom values.

Presumably the protein composition is of importance for the lower gel points in fish gelatin grades. The proportions of the amino acids proline and hydroxyproline are considerably lower in the case of fish gelatins, and specifically especially in the case of cold water fish gelatins, compared with pig, cattle and poultry gelatins. Hydroxyproline and proline play an important role in the cross-linking of the protein helices with one another. Presumably folding of the helices takes place in water, water can be embedded and the solubility rises. The temperature at which this folding takes place depends on the hydroxyproline content and proline content. The lower the content, the lower the temperature at which the gelatin goes into solution. It has been determined that a low gel temperature and a low solubility temperature is advantageous for good solubility in the mouth.

As a rule it is not possible to achieve adequate process stability of the coating-free capsule by using fish gelatin having a Bloom value of less than 200 as the only type of gelatin in the shell. The gel strength of the coating-free capsule shells that are still moist is frequently not adequate for further processing. The coating-free capsules are frequently mechanically too unstable.

On the other hand, the particular technical problem is solved by the admixture of such a gelatin, as readily soluble filler, to a high-Bloom gelatin that is intended to form a process-stable skeleton. In this context mixtures of fish gelatins with Bloom values of below 200 and high-Bloom pig, cattle or poultry gelatins with a Bloom value of over 200 have proved advantageous. Fish gelatin (below 200 Bloom) contents of 0.5-50% (m/m), based on the solids content of the shell, are preferred. In this context gelatin grades from cold water fish are particularly preferred. Here it is the gel point of the high-Bloom gelatin fraction that is the decisive factor determining the gel point of the mixtures.

### Plasticizers:

Plasticizers that can be used in the preparation of the shell are, in particular, polyols, such as, for example, sorbitol, glycerol, propylene glycol, lactitol, hydrated hydrolyzed starches and trehalose. Plasticizer fractions improve the consumption characteristics of a capsule in that they reduce the hardness of the shell of the coating-free capsule and improve the solubility in the mouth. Moreover, plasticizers promote the flexibility of the shell and thus the stability during drying of the coating-free capsule and during transport.

Preferred plasticizer contents are not more than 30% (m/m) based on the total solids content of the shell. Higher amounts of plasticizer make drying of the coating-free capsules more difficult and also make it necessary to use packaging that excludes atmospheric humidity.

Plasticizers are preferably used in the shell in a proportion of 10-30% (m/m), particularly preferentially of 15-20% (m/m), based on the solids content of the shell. The plasticizer preferably comprises one or more polyols, preferably selected from the group which consists of glycerol, propylene glycol, sorbitol and maltitol. Glycerol is the preferred plasticizer.

Plasticizer contents of over 30% (m/m) make drying of coating-free capsules disclosed herein more difficult and frequently make it necessary to use anti-caking agents, such as silica. In contrast, plasticizer contents of less than 10% (m/m) allow the capsule shell of a coating-free capsule to become increasingly brittle.

### Further (Optional) Constituents of the Capsule Shell:

### Sweeteners/Colorants/Acids/Water/Breath Control Actives.

In addition to gelatin and plasticizer, the shell of the coating-free capsule disclosed herein can contain sweeteners, such as, for example, sucralose, aspartame, acesulfame, K or Na saccharine, thaumatin, neohesperidin, or mixtures thereof, as well as water-soluble food colorants.

In addition to the materials mentioned above, the shell of a coating-free capsule can contain acids, in particular of the monoacid, diacid or triacid type, preferably citric acid, fumaric acid, malic acid, lactic acid or acetic acid. The use of at least one such acid makes it possible, in particular, to ensure microbiological stability of the shell of the coating-free capsule and to tailor its sensory properties. In addition the physicochemical properties can be adjusted (pH, solubility).

The coating-free capsules are dried during the production process. During this operation a certain residual amount of water remains bound in the gelatin network. Depending on the ambient moisture content, a water content will be established in the coating-free capsule shell in equilibrium. At 20° C. and 50% relative atmospheric humidity, the equilibrium moisture content of typical coating-free capsules described herein is in the range of approximately 8-10% (m/m) water, based on the total mass of the coating-free capsule shell.

Breath control actives, suitable for inclusion in the shell of the coating-free capsules are quaternary compounds such as pyridinium salts (e.g., cetyl pyridinium chloride), other cationic materials such as chlorhexidine salts, zinc salts, surfactants such as sodium lauryl sulfate, salts such as sodium laurate, chlorophyll, triclosan, copper compounds such as copper gluconate or copper-chlorophyll-extract.

### Hydrocolloids/Gellan Gum:

Additions of hydrocolloids to gelatin influence the solubility and thus the absorption of water as well as the temperature stability of the gels formed. The hydrocolloid gellan gum, in particular, can advantageously be used as an admixture to the gelatin in a shell material mixture for the preparation of a coating-free capsule according to the invention. Gellan gum is a gel-forming polysaccharide that is prepared by fermentation with the aid of microorganisms.

U.S. Pat. No. 4,517,216 already describes mixtures of gelatin and gellan gum. By means of a gellan gum content of 16%-83%, based on the sum of the gellan gum amount and gelatin amount, a high gel strength of the shell is achieved as a result of a synergistic effect. It is also described that only the deacylated and partially deacylated forms of gellan gum give rise to this effect, but not the native gellan gum.

If the multi-component nozzle method with immersed nozzle is to be used for the preparation of a coating-free capsule according to the invention, it is essential when using gellan gum in the shell material to make the correct choice of the gellan gum type and the gellan gum amount so that the shell does not solidify even before formation of the coating-free capsule has been completed. Moreover, the viscosity of the shell solution must not be too high.

In order to achieve an increased temperature stability, gellan gum is advantageously added to the shell mixture for the preparation of a coating-free capsule according to the invention; as a result the softening temperature of the shell increases considerably and the gelling temperature of the mixture is also considerably increased. For the preparation of coating-free capsules according to the invention by the multi-component nozzle method with immersed nozzle, the gelling temperature should not be above 50° C. and the viscosity of the shell solution at 80° C. should not be above 300 mPas; otherwise formation of the coating-free capsule is made more difficult or is not achievable. Therefore, the gellan gum type and amount must be selected in a particularly targeted manner.

There are high-acylated and low-acylated gellan gum grades. A low-acylated gellan gum, preferably the KELCOGEL F grade from Kelco, a division of Merck & Co, is preferably used for the preparation of coating-free capsules according to the invention. Hard, transparent gels can be obtained using low-acylated gellan gum grades.

The preparation of coating-free capsules used in the present invention by the multi-component nozzle process is problematical with a high-acylated gellan gum grade, such as, for example, KELCOGEL LT100 from Kelco, since during capsule shaping the coating-free capsules do not release from the coaxial nozzle without disturbance because of high elasticity of the shell. Moreover, undesired turbid and very highly elastic soft gels are produced.

In a preferred aqueous shell mixture (shell solution) for the preparation of a coating-free capsule according to the invention gelatin, with a content of >15% (m/m), based on the total mass of the shell solution, makes up the major proportion of hydrocolloids used in the total mass. In addition, gellan gum is used in a proportion of at most 0.6% (m/m), preferably a proportion in the range between 0.2 and 0.5% (m/m).

Higher proportions of gellan gum substantially increase the viscosity of the shell solution during capsule shaping and substantially reduce the solubility of the dried coating-free capsule shell in the mouth, which is not desired.

Lower proportions of gellan gum have no particular effect with regard to improved temperature stability of the dried coating-free capsule. The presence of gellan gum leads to the formation of a solid network in the shell of a coating-free capsule according to the invention, which solid network as a rule does not dissolve in the moist state, even at 40-60° C. This network should make up only a relatively small proportion of the shell as a strengthening element. The fractions of non-crosslinked gelatin and further additives such as, for example, plasticizers, should, on the other hand, dissolve particularly rapidly in the mouth.

Coating-free capsules described herein can contain gellan gum in a range of 0.4-3% (m/m), preferably of 0.8-2% (m/m), based on the solids content of the shell are preferred. A preferred mass ratio of gellan gum to gelatin in the range of 1:23 to 1:230, preferably of 1:35 to 1:115 is obtained with gelatin contents of 70-90% (m/m), based on the solids content of the shell.

The gelatin fraction in these coating-free capsules can, in particular, also contain fractions of 0-Bloom gelatin and/or low-Bloom fish gelatin (in this context see above).

### EXAMPLES

Although not intending to limit the scope of the invention, Applicants provide the following examples of coated capsule delivery systems for the delivery of nutraceutical actives in order to better describe the invention. Exemplary coated capsules disclosed herein that provide antacid efficacy while producing a desirable flavor can be found in Examples 1-4.

| | | | |
|---|---|---|---|
| Example 1 | Example 2 | Example 3 | Example 4 |

CORE (inner capsule)

| | | | | |
|---|---|---|---|---|
| Vegetable oil | 150 | 20 | 100 | 150 |
| Flavor | 50 | 150 | 50 | 50 |
| Cooling agent | | 30 | — | — |
| Sweetener | 1 | | | |
| (disp.) | | | | |
| Vitamin D | 0.005¹⁾ | 0.005¹⁾ | — | — |
| Vitamin E | — | — | 50²⁾ | — |

SHELL (inner capsule)

| | | | | |
|---|---|---|---|---|
| Gelatin | 25 | 25 | 25 | 25 |
| Plasticizer | 24 | 24 | 24 | 24 |
| Sweetener | 1 | 1 | 1 | 1 |

PROTECTION LAYER

| | | | | |
|---|---|---|---|---|
| Gum | 0.1 | 0.1 | 0.1 | 0.1 |
| Fat (TAG) | — | 0.2 | — | — |

COATING

| | | | | |
|---|---|---|---|---|
| Sugar/Sugar | 470 | 470 | 400 | 470 |
| alcohol | | | | |
| Calcium | 480³⁾ | 480³⁾ | 1250⁴⁾ | — |
| carbonate | | | | |
| Antacid | — | — | — | 470 |
| Wax | 0.1 | 0.1 | 0.1 | 0.1 |

Values per capsule are in milligram (weight abs.).
¹⁾Corresponds with 200 I.U. vitamin D per capsule, equaling 20% of the RDA.
²⁾Equaling 25% of the RDA per capsule.
³⁾Corresponds with 188 mg Calcium per capsule, equaling 20% of the RDA.
⁴⁾Corresponds with 500 mg Calcium per capsule, equaling 50% of the RDA.

The foregoing is provided for purposes of illustrating, explaining, and describing embodiments of this invention. Modifications and adaptations to these embodiments will be apparent to those skilled in the art and may be made without departing from the scope or spirit of this invention.

What is claimed is:

1. A coated spherical capsule comprising:
a seamless capsule, comprising a seamless solid shell surrounding a mononuclear liquid or viscous core; and
an edible coating surrounding said seamless capsule, said edible coating comprising at least one dietary supplement and at least one sugar or sugar alcohol,
wherein said liquid or viscous core comprises 1-100 wt-% of a flavoring, based on total weight of the liquid or viscous core, and wherein the edible coating comprises an effective amount of said at least one dietary supplement based on a weight of the edible coating.

2. The coated spherical capsule according to claim 1, wherein the edible coating comprises 5-70 wt-% of said at least one dietary supplement based on the weight of the edible coating.

3. The coated spherical capsule according to claim 1, wherein said edible coating comprises 15-40 wt-% of at least one bulk mineral, at least one trace mineral, or both.

4. The coated spherical capsule according to claim 3, wherein the coated spherical capsule further comprises at least one oil soluble compound that enhances absorption of the at least one mineral, the at least one trace mineral, or both.

5. The coated spherical capsule according to claim 1, wherein said edible coating comprises an effective amount of at least one bulk mineral selected from the group consisting of calcium, zinc, iron, magnesium, potassium, phosphorous, sodium, ions thereof, and combinations thereof.

6. The coated spherical capsule according to claim 1, wherein the liquid or viscous core comprises at least one oil soluble vitamin or pro-vitamin.

7. The coated spherical capsule according to claim 6, wherein said at least one oil soluble vitamin or pro-vitamin is selected from the group consisting of Vitamin E, Vitamin K, Vitamin A, beta-carotene, carotenoids, and combinations thereof.

8. The coated spherical capsule according to claim 1, wherein said edible coating comprises at least one water-soluble vitamin.

9. The coated spherical capsule according to claim 8, wherein said water-soluble vitamin is selected from the group consisting of Vitamin B, Vitamin C, and combinations thereof.

10. The coated spherical capsule according to claim 1, wherein the edible coating comprises a mixture of (i) Zinc with Vitamin C, (ii) Calcium with Vitamin D, (iii) Selenium with Vitamin B12, or (iv) a combination thereof.

11. The coated spherical capsule according to claim 1, wherein the coated spherical capsule comprises at least 20% of the Dietary Reference Intake (RDI) or the Recommended Dietary Allowance (RDA) of at least one dietary supplement.

12. The coated spherical capsule according to claim 1, comprising at least one botanical extract.

13. The coated spherical capsule according to claim 12, wherein the at least one botanical extract comprises a botanical extract selected from the group consisting of Echinacea, St John's Wort, Ginseng, Tea polyphenols, extracts thereof, and combinations thereon.

14. The coated spherical capsule according to claim 1, comprising at least one therapeutic agent for:
(i) treating acid reflux disease or symptoms thereof,
(ii) providing an anti-inflammatory effects,
(iii) providing an antiseptic effect,
(iv) providing an analgesic effect, or
(v) a combination thereof.

15. The coated spherical capsule according to claim 14, wherein the at least one therapeutic agent comprises a therapeutic agent selected from the group consisting of salts of bismuth, magnesium, aluminum, calcium, and combinations thereof.

16. The coated spherical capsule according to claim 1, wherein the coating comprises at least two layers comprising different dietary supplements, different compounds, or both.

17. The coated spherical capsule according to claim 1, wherein the coating comprises at least two layers comprising different dietary supplements, wherein said different dietary supplements will react with one another, and wherein said at least two layer are separated by a third layer preventing said different dietary supplements from reacting with one another in coated spherical capsule form.

18. A method of administering an effective amount of a dietary supplement comprising:
providing a plurality of coated spherical capsule according to claim 1;
ingesting a quantity of said coated spherical capsules on at least two consecutive days in order to deliver an effective amount of at least one dietary supplement present in said coated spherical capsules.

19. The method according to claim 18, wherein said at least one dietary supplement is a bulk mineral selected from the group consisting of calcium, zinc, iron, magnesium, potassium, phosphorous, sodium, ions thereof, and combinations thereof.

20. The method according to claim 18, said ingesting step includes ingesting a quantity of said coated spherical capsules in at least two consecutive days in order to deliver at least 50% of a Dietary Reference Intake (RDI), a Recommended Daily Allowance (RDA), or both, of at least one dietary supplement present in said coated spherical capsules.
